(19) United States
(12) Patent Application Publication
(10) Pub. No.: US 2026/0127739 A1
(43) Pub. Date: May 7, 2026

(54) METHOD FOR ADAPTING DEPTH STRUCTURE MODEL DATA TO COHERENCE TOMOGRAPHY MEASUREMENT DATA AND COHERENCE TOMOGRAPHY METHOD

(71) Applicant: Friedrich-Schiller-Universitaet Jena, Jena (DE)

(72) Inventors: Felix WIESNER, Bonn (DE); Silvio FUCHS, Braunsdorf (DE); Gerhard G. PAULUS, Jena (DE); Johann Jakob ABEL, Dornburg-Camburg (DE)

(73) Assignee: Friedrich-Schiller-Universitaet Jena, Jena (DE)

(21) Appl. No.: 19/382,020

(22) Filed: Nov. 6, 2025

Related U.S. Application Data
(63) Continuation of application No. PCT/EP2024/062672, filed on May 8, 2024.

(30) Foreign Application Priority Data
May 8, 2023 (DE) 10 2023 111 920.6

(51) Int. Cl. G06T 7/00; G06T 5/10; G06T 7/50; G16H 30/20

(52) U.S. Cl. CPC G06T 7/0012; G06T 5/10; G06T 7/50; G16H 30/20; G06T 2207/10101; G06T 2207/30041

(57) ABSTRACT

A method for adapting depth structure model data to coherence tomography measurement data of a sample having a plurality of layers composed of layer materials in a layer construction in a depth direction. A plurality of layers having layer thicknesses and a plurality of layer transitions upstream of the respective layers in the depth direction are derived from the depth structure. Furthermore, model reflectivity coefficients are adapted to the respective layer-transition-specific, spectrally resolved reflectivity coefficients by way of a value that optimizes the adaptation being calculated in each case for the at least one parameter of one of the model layer transitions. On the basis of the value calculated for the at least one parameter, depth structure model data can be obtained, the resolution of which in the depth direction goes beyond the resolution predefined by a CT measurement on which the spectral complex field reflectivity is based.

### CROSS REFERENCE TO RELATED APPLICATIONS

This application is a continuation under 35 U.S.C. § 120 of International Application PCT/EP2024/062672, filed May 8, 2024, which claims priority to German Application No. 10 2023 111 920.6, filed May 8, 2023, the contents of each of which are incorporated by reference herein.

The present invention relates to a computer-implemented method for adapting depth structure model data to coherence tomography measurement data of a sample and to a method for examining a sample by means of optical coherence tomography.

Optical coherence tomography (OCT) has become established as an optical microscopy method for diffraction-free three-dimensional imaging. OCT is based on backscattering of incident light at an interface of a sample to be examined. The interface is also referred to herein as a layer transition. Information about the sample to be examined can be obtained, for example, by evaluating the interference of the light (back)scattered at different interfaces. In addition to the spatial superposition, a low temporal coherence of the superimposed light is a precondition. See “Optical coherence tomography—principles and applications”, A F. Fercher et al., Rep. Prog. Phys. 66 239 (2003) for a fundamental introduction into OCT.

The spectral range for OCT investigations was extended to the spectral range from 30 eV to 530 eV within the scope of the so-called XCT (optical coherence tomography in the XUV range) using extreme ultraviolet (XUV) sources (e.g. synchrotron-based or laser-based). Siee, for example, U.S. Pat. No. 7,656,538 B2 (“short-wave-length coherence tomography”) for a laser-based XCT. Due to the large usable frequency bandwidths in the XUV, XCT enables an axial resolution in the range of, e.g., a few tens of nanometers. This results in new applications in the non-destructive examination of, e.g., (multilayer) coatings such as optical or XUV mirrors, functional axial structures in solar cells or axially structured semiconductors (graphene-based electronics) and of biological membrane layer structures. In particular, XCT enables a three-dimensional imaging of near-surface structures of thick samples. In the examination of structured semiconductors, XCT represents, for example, an interesting alternative to scanning electron microscopy (SEM) or transmission electron microscopy (TEM) as the latter usually require a preparation of the sample which often destroys the sample.

In the scientific publication “Laboratory setup for extreme ultraviolet coherence tomography driven by a high-harmonic source”, J. Nathanael et al., Rev. Sci. Instrum. 90, 113702 (2019), an exemplary setup for the detection of XCT spectra using the example of a nanostructured sample and an exemplary data processing of the XCT spectra are described. Further introductory explanations for spectroscopic XCT are furthermore disclosed in “Coherence tomography with broad bandwidth extreme ultraviolet and soft X-ray radiation”, S. Skruszewicz et al., Applied Physics B (2021) 127:55.

How an exemplary XCT algorithm in combination with a one-dimensional phase retrieval (PR) algorithm can derive the depth structure of a sample from the autocorrelation signal is explained in detail in the scientific publication “Optical coherence tomography with nanoscale axial resolution using a laser-driven high-harmonic source”, S. Fuchs et al., Vol. 4, No. 8/August 2017/Optica and the associated supplementary information.

In the scientific publication “Characterization of encapsulated graphene layers using extreme ultraviolet coherence tomography”, F. Wiesner et al., Vol. 30, No. 18/29 Aug. 2022/OpticsExpress 32267, it is furthermore disclosed how ratios of reflectivities following an XCT depth structure analysis can be used for model-based acquisition of boundary layer parameters.

The inventors have recognized that OCT is limited in its resolution in the depth direction by the bandwidth of the incident light, wherein, however, structures, which cause the backscattering of the incident light, can have a size in the depth direction that is below the OCT resolution given by the bandwidth.

An aspect of this disclosure is based on the object of providing a method that can resolve structures of a sample within the scope of a coherence tomography (CT), which are below the resolution of the coherence tomography given by the bandwidth. A further object is to derive parameters of such structures and to adapt a depth structure model of the sample based thereon. Furthermore, aspects of the disclosure can be based on the object of characterizing layer transitions in a sample having a layer structure in more detail.

At least one of these objects is achieved by a method according to claim 1 or by a method according to claim 10. Developments are provided in the dependent claims.

In one aspect, a computer-implemented method for adapting depth structure model data to coherence tomography (CT) measurement data of a sample, which—in a depth direction—has a plurality of layers (Si) of layer materials in a layer structure, comprises the following steps:
providing CT measurement data in a processor, wherein the CT measurement data comprise a depth structure r(z) unambiguously derived from a spectral complex field reflectivity, wherein the depth structure r(z) is based on a resolution in the depth direction, which is predetermined by a CT measurement on which the spectral complex field reflectivity is based;
(in particular by data processing based on the depth structure r(z) using the processor) deriving from the depth structure r(z) a plurality of layers having layer thicknesses and a plurality of layer transitions, which are arranged upstream of the respective layers in the depth direction; (It is noted that the positions/region of the layers and layer transitions in the depth direction result from the layer thicknesses of the layers in the layer sequence.)
(in particular by data processing based on the depth structure r(z) using the processor) extracting layer transition-specific, spectrally resolved reflectivity coefficients from the depth structure r(z) for the plurality of layer transitions;
providing depth structure model data in the processor, wherein the depth structure model data model the layer structure of the sample and are initialized for the plurality of derived layers based on the layer thicknesses and the layer materials, which belong to the layers, and a plurality of model layer transitions, the spectrally resolved model reflectivity coefficients of which can be modeled in a layer transition-specific manner based on at least one parameter; and
(in particular by data processing using the processor) adapting the model reflectivity coefficients to the respective layer transition-specific, spectrally resolved reflectivity coefficients by calculating a value optimizing the adaptation respectively for the at least one parameter of one of the model layer transitions; and
(in particular by data processing using the processor) adapting the depth structure model data on the basis of the value calculated for the at least one parameter (and in particular storing the adapted depth structure model data in a memory and/or outputting the adapted depth structure model data on a display).

In some embodiments of the method, before adapting a model reflectivity coefficient of a layer transition, the model reflectivity coefficients, which precede respectively in the depth direction, can be adapted to the layer transition-specific, spectrally resolved reflectivity coefficients.

In some embodiments of the method, the depth structure model data can be adapted layer by layer in the depth direction with respect to the layer transitions. Additionally or alternatively, the value calculated for the at least one parameter can be below the resolution in the depth direction preset by the CT measurement data.

In some embodiments, the model reflectivity coefficients for adapting the depth structure model data to the CT measurement data can be recursively optimized in a plurality of steps with respect to layer transitions located deeper in the sample, wherein, in particular, starting from the surface of the sample, the layer structure can be reconstructed and the depth structure model data can be updated layer by layer by
in a first step, parameters of a surface layer (such as a roughness and a thickness of the surface layer) are optimized by minimizing a deviation between measured and modeled reflectivity coefficients of the surface layer by varying the parameters, and the values of the parameters of the surface layer are fixed in the depth structure model data for the further optimization;
in a second step, parameters of the second layer transition in the depth direction, such as a roughness and a thickness of a buried oxide layer, are optimized by minimizing a deviation function between measured and modeled reflectivity coefficients of the second layer transition by varying the parameters while the parameter of the surface layer being fixed, and the values of the parameters of the second layer transition are fixed in the depth structure model data for the further optimization; and
in further steps, the parameters of the layer transition respectively closest in the depth direction are successively optimized (and in particular fixed for subsequent adaptations). Optionally, the sum of the thicknesses determined for the surface layer and the subsequent layer transitions and the thicknesses of the layers determined from the depth structure r(z) can be kept constant with respect to the depth structure r(z).

In some embodiments of the method, layer transition-specific, spectrally resolved reflectivity coefficients can be extracted by:
identifying a depth region in the depth structure r(z) assigned to a layer transition, in particular by sequential spatial filtering of the depth structure r(z); and
calculating the layer transition-specific, spectrally resolved reflectivity coefficients by a Fourier transformation of the field reflectivity r(z) limited to the respective layer transition.

In some embodiments of the method, the at least one parameter of a model reflectivity coefficient of a layer transition can be selected from the group of parameters comprising a layer transition thickness, a roughness, a spectral reflection or transmission value, a material density, a material type, a material composition, a stoichiometric ratio of a material composition, and/or a material transition gradient of a material composition.

In some embodiments of the method, the layer transition can comprise an interface or an intermediate layer delimited by two interfaces between two layers of the same material or an interface or an intermediate layer delimited by two interfaces between two layers of different materials.

In some developments, the method can further comprise a first step of adapting the initialized depth structure model data to the coherence tomography measurement data, which comprises optimizing a model reflectivity coefficient, which is assigned to an input-side interface of the sample and represents a first modelable layer transition, in particular a modelable surface layer, by matching with a reflectivity coefficient.

In some developments, the method can further comprise an output step, in which the adapted depth structure model data and in particular the set values of the at least one parameter for the layer transitions are output by the processor on an input and output device (and alternatively or additionally stored by the processor in a memory).

In some embodiments of the method, the value optimizing the adaptation for a layer transition can be calculated by, within the scope of a parameter scan, minimizing a deviation function, which is dependent on the at least one parameter, between layer transition-specific, spectrally resolved reflectivity coefficients assigned to the layer transition and the model reflectivity coefficient assigned to the layer, in particular iteratively.

In a further aspect, a method for examining a sample, which has a plurality of layers in a layer structure in a depth direction, using optical coherence tomography, comprises the following steps:
performing a CT measurement on the sample, wherein a spectral complex field reflectivity is generated as CT measurement data by a phase-sensitive measurement and evaluation of an optical coherence signal or a spectrally resolved measurement, performed in particular within the scope of an artifact-free coherence tomography data analysis, of an intensity reflectivity of the sample and a derivation of phase information by an iterative phase retrieval method;
deriving an unambiguous depth structure r(z) by Fourier transforming the spectral complex field reflectivity using a processor, wherein the depth structure is based on a resolution in the depth direction, which is predetermined by the CT measurement;
(in particular by data processing based on the depth structure r(z) using the processor) deriving layers and layer thicknesses of the sample taking into account a dispersion correction based on known layer materials of the sample;
generating depth structure model data using the processor, wherein the depth structure model data model the layer structure of the sample and are initialized for the plurality of derived layers based on the layer thicknesses and the layer materials, which belong to the layers, and a plurality of model layer transitions, the spectrally resolved model reflectivity coefficients of which can be modeled in a layer transition-specific manner based on at least one parameter; and
performing a computer-implemented method, in particular as described above, for adapting the depth structure model data to the CT measurement data of the sample, whereby the depth structure model data are expanded in particular by parameters of model layer transitions.

In some embodiments of the method, the CT measurement on the sample can be performed using XUV radiation; and in particular, the spectral width of the XUV radiation can limit a resolution of the CT measurement in the depth direction to a range of a few 10 nanometers, and the computer-implemented method can identify structures assigned to the transition layers with a resolution in the nanometer range.

In some embodiments of the method, the CT measurement on the sample can be performed for a plurality of lateral regions, in particular in order to detect lateral inhomogeneities, which are present in a layer, of parameters of the layer structure. In particular, radiation can be irradiated successively onto the lateral regions, such that a spectral complex field reflectivity is respectively generated for the lateral regions of the plurality of lateral regions for performing the computer-implemented method for adapting the depth structure model data to the CT measurement data of the sample.

The concepts described herein relate to the depth structure r(z) of a sample (also referred to as an axial sample structure). As is evident, inter alia, from the publications mentioned above, in the context of coherence tomography, a depth structure r(z)—that is to say a “reflective” response function—results from the measurement of the backscattering of the incident light. DiThe depth structure r(z) represents a distribution of the complex reflectivity (derived from the measurement) along the depth direction z. The depth structure r(z) (i.e., the axial distribution of the complex reflectivity) can be evaluated in order to describe the measured sample using a layer structure model. The evaluation can result, e.g., in axial interface positions, i.e., interface positions given in the depth direction; an interface is also referred to herein as a layer transition. An interface/a layer transition can have, e.g., a specific interface reflectivity (e.g., increased in comparison with adjacent “homogeneous” material regions). A (material) layer can be assigned to the region between two adjacent interface positions in the layer structure model, wherein the position and the extent of the layer in the depth direction are given by the position of the interfaces. Thus, layer parameters of “thick” layers of the structure of a sample can be derived from the depth structure r(z) (derived from the measurement). The layer structure model assigned to the sample can be constructed from the position and the extent of the layers.

The concepts described herein can have, among other things, the following advantages over the prior art in the generation of models of a sample (such as electron microscopy) or avoid corresponding disadvantages of the prior art: a non-destructive interaction with the sample, essentially no need for a destructive sample preparation, a low heat input during the measurement, a lateral resolution in the nanometer range as well as high sampling rates, in particular in the case of XCT. Advantages of the laser-based XCT methods exemplarily disclosed herein can be transferred at least in part to OCT methods in the visible or infrared spectrum.

Using the computer-implemented method disclosed herein for adapting depth structure model data to CT measurement data of a sample, an (essentially non-destructive) XCT examination can measure a layer structure and a layer composition of a sample with a spatial depth resolution, which corresponds to that of electron microscopy or can even be higher. At the same time, a determination of the roughness of outer and inner interfaces can be enabled with the disclosed methods.

A sample characterization based on the computer-implemented method disclosed herein using a novel, model-based step-by-step optimization in the reconstruction of a layer structure, which can be combined, inter alia, with broadband extreme ultraviolet (EUV) and soft X-ray radiation for a CT measurement, can provide information about local material parameters within the scope of a highly precise depth structure analysis with nanometer resolution. Such determinable material parameters comprise, e.g., a stoichiometric ratio of a material composition, a thickness and/or a roughness of a layer transition, be it at a surface (outer interface) or at an internal interface. In general, based on the method disclosed herein, conclusions can be drawn about existing material phases in a sample and their spatial dimensions, for example, a thickness of existing native surface oxide layers and interfacial oxide layers.

A further advantage is that a limitation of the Fourier transformation (“truncated Fourier transform”) applied in the method in depth direction can reduce when extracting spectrally resolved reflectivity coefficients of layer transitions—quasi as a filtering process—the noise in the layer transition-specific measurement data. In the frequency domain, the truncated Fourier transformation acts like a sharp frequency filter, such that only the modulation of the depth structure r(z) is evaluated, which corresponds to a layer transition that is considered within the scope of an optimization step. Thus, the proposed method offers the advantage of being able to more precisely determine physically measurable variables by individual sequential optimization steps and, thus, already to include them in the sample model for further optimization steps. Conceptually, this is an essential difference to, for example, an OCT brute force optimization approach with completely free parameters and only one measured variable, the OCT depth structure.

Herein, concepts are disclosed that allow aspects from the prior art to be improved at least in part. In particular, additional features and their practicality result from the following description of embodiments with reference to the figures. Of the figures:

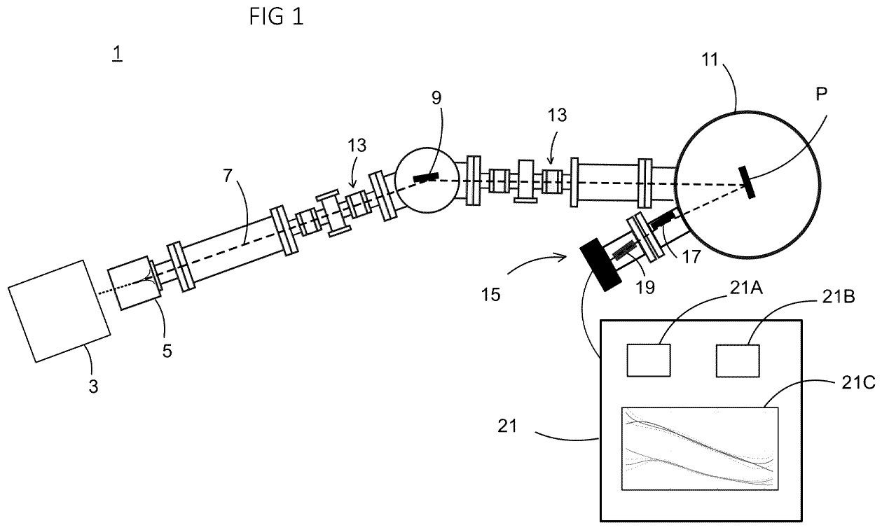

FIG. 1 shows a schematic representation of an exemplary measurement setup for a coherence tomography measurement with broadband extreme ultraviolet (EUV) and soft X-ray radiation;

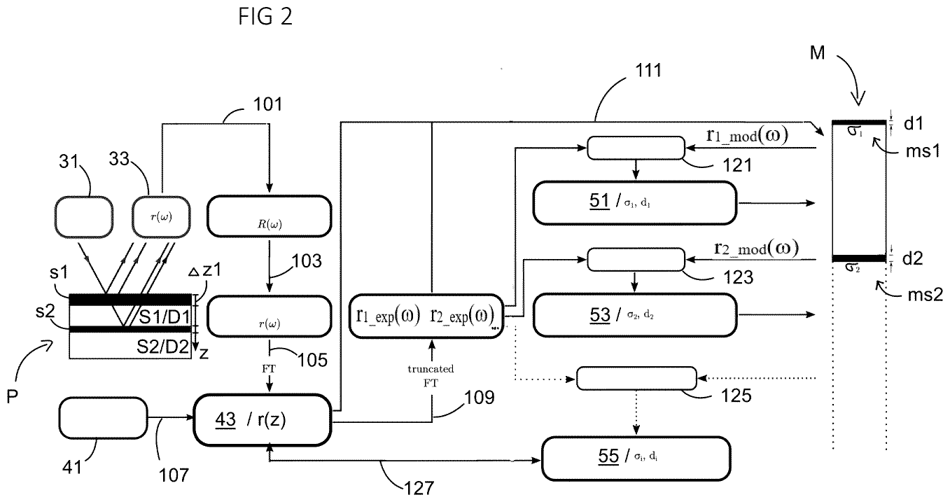

FIG. 2 shows a flow diagram for illustrating an exemplary method for examining a sample by means of a coherence tomography measurement and its exemplary evaluation of measurement data according to the invention;

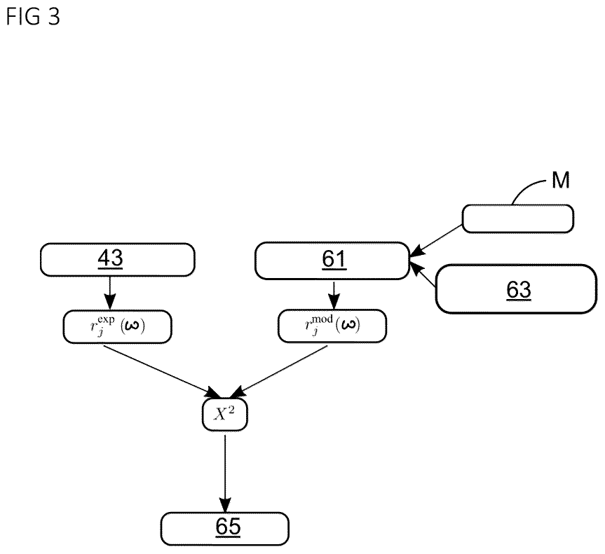

FIG. 3 shows a flow diagram for illustrating an exemplary calculation of a parameter of a modeled layer transition during the optimization of a spectrally resolved model reflectivity coefficient with respect to a measured reflectivity coefficient; and

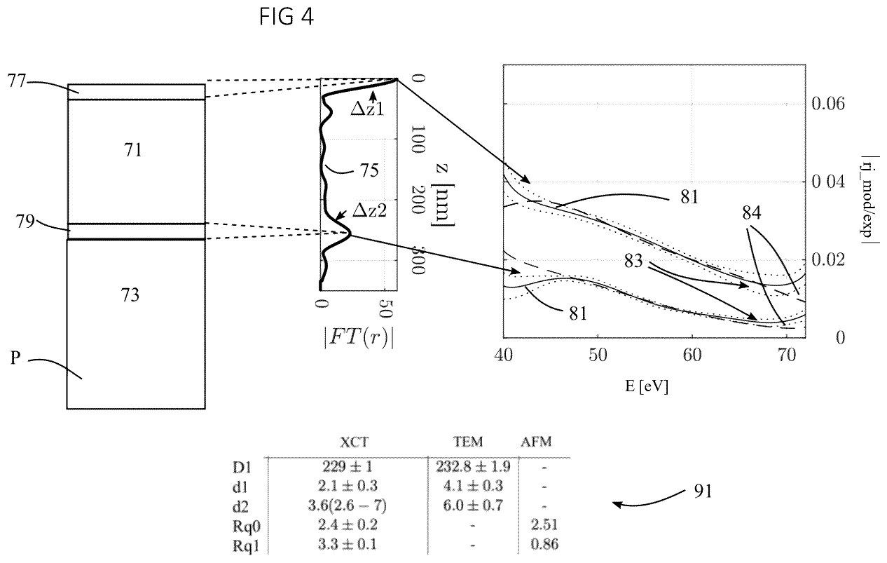

FIG. 4 shows a sketch for illustrating an adapted depth structure model.

It is essential for the concepts described herein that a measured complex field reflectivity r(ω) comprises information with respect to a structure of a sample, which comprises layers, as well as phase information with respect to spectrally resolved reflectivity coefficients r_{i_exp}(ω), which are respectively assigned to a layer transition.

For an adaptation of depth structure model data to CT measurement data, the inventors use in particular the possibility of recursively comparing experimentally measured reflectivity coefficients of the layer transitions r_{i_exp}(ω) with the model reflectivity coefficients r_{i_mod}(ω) resulting from the depth structure model data. Thereby, the inventors have recognized in particular that dispersion and absorption/reflectance in layers located thereabove, through which radiation passes beforehand, have an effect on the radiation reflected by a layer transition. Starting from this, for the creation of the depth structure model data, a recursive calculation of the reflectivities of the layer transitions is proposed that begins at the incidence side (sample surface) and continues layer by layer into the sample. This procedure simplifies the calculations that are performed for the optimization of depth structure model data. In other words, the inventors propose a recursive optimization of the depth structure model data that, modeled after the physical reflection process in the sample, implements the dependence of the individual layer reconstructions on one another not only on the level of the modeling of the model reflectivity coefficients r_{i_mod}(ω), but also in the sequence of the reconstruction, in particular within the scope of an iterative adaptation.

Furthermore, the inventors have recognized that for a reconstruction of parameters of a layer transition, inter alia, the influences of roughness and layer thickness on the reflectivity of a layer transition can be distinguished. This has an effect in particular on the modeling of the reflectivities of the layer transitions and their optimization with respect to the measurement data.

In general, the starting point of the method disclosed herein is an OCT/XCT measurement that, in addition to the depth structure information, provides phase information about the reflections at layer transitions for the evaluation of the measurement data, for example, in the form of a spectral complex field reflectivity r(ω) of the sample. Exemplary approaches of OCT measurement methods that capture phase information are:

(1) OCT measurement methods that measure the phase of the sample reflectivity in an interferometric setup relative to a reference object of known phase and reflectivity, e.g., within the scope of a spatial or spectral scanning process.

(2) Fourier-domain OCT measurement methods (in particular XCT measurement methods) that have been supplemented by an evaluation of the phase, e.g., with a one-dimensional phase retrieval (PR) algorithm.

The aspects of the evaluation described herein relate in particular to the so-called Fourier-domain OCT variant (FD-OCT/FD-XCT). This variant differs from the time-domain OCT variant that is implemented, e.g., within the scope of an axial scanning of the sample, e.g., by the movement of a reference mirror. The time-domain OCT variant cannot be implemented technically, or can only be implemented technically with difficulty, for XCT so that XCT is usually implemented as spectrometer-based OCT (also referred to as FD-OCT)—as explained below in connection with FIG. 1—or alternatively as swept-source OCT. In the case of FD-OCT, the actual measured variable is the spectrum reflected by the sample, which is modulated and carries the information about the axial structure of the sample (depth information).

FIG. 1 schematically shows an XCT measurement setup 1 for XUV coherence tomography. The setup comprises a radiation source 3, for example, a femtosecond laser system for generating pulsed laser radiation with a central wavelength of 800 nm, pulse durations in the range of a few tens of femtoseconds at repetition rates in the kHz range. The wavelength can be shifted into the infrared in an optical parametric amplifier. The infrared laser pulses with a pulse energy in the mJ range are focused in a source chamber 5, e.g., in argon gas, for high-harmic generation (HHG). By changing the delay between pump pulse and signal pulse in the optical parametric amplifier, XUV radiation with a quasi-continuous spectrum can be generated, which propagates along an optical beam path 7 of the XCT measurement setup.

The XUV radiation generated in the source chamber 5 is focused by a toroidal mirror 9 (e.g., f=1 m) onto a sample P in a sample chamber 11. From the XUV generation, the optical beam path 7 of the XUV radiation runs in vacuum, wherein pressures in the range of, e.g., ^{˜}10⁻⁸ mbar can be present using differential pump stages in a sample chamber 11. Infrared radiation components can be filtered by thin aluminum and zirconium membranes 13.

Furthermore, the sample P and the beam path 7 can be displaced relative to one another (for example, by moving the sample P by means of a controllable sample holder, in particular in a (lateral) direction orthogonal to the normal of the sample surface or to the direction of incidence of the XUV radiation). This enables measurements to be performed at different lateral regions (positions) of the sample P. In other words, the beam path 7 can enter the sample P at different positions on the surface, such that different regions of a layer can be scanned and, e.g., lateral inhomogeneities of sample parameters can be detected (for example, a layer thickness, a roughness of a layer transition, etc.).

Radiation reflected by the sample P is measured by means of a spectrometer 15. For example, the radiation focused in one plane by a cylindrical mirror 17 can be spectrally characterized efficiently in the other plane using a 1200-line grating 19. In the detected spectrum, interferences due to the reflection at a layer structure of the sample P are present, which allow the characterization of the sample P with respect to the layer structure in an evaluation unit 21.

To separate the incident and the emergent radiation, the sample P is illuminated at an angle. In the evaluation of the detected spectrum, the unavoidable absorption of the XUV radiation propagating in the sample P plays an essential role, especially also in the XUV range. The strength of the absorption (and thus the transmission of the radiation to a deeper layer transition) depends on the frequency of the radiation and the materials being present in a sample P and, thus, has to be taken into account in the modeling of the reflectivities of layer transitions present in the sample P in the depth structure model.

The evaluation unit 21 can comprise a central processing unit (CPU), which is programmed to carry out calculations in connection with the adaptation of depth structure model data to coherence tomography measurement data by executing instructions stored in program code. The CPU can comprise a microprocessor 21A or a plurality of microprocessors in connection with a memory element 21B or a plurality of memory elements as well as an input and output device 21C (e.g., a touchscreen). The memory element 21B can store one or more microprocessor-readable instructions (program code), CT measurement data and depth structure model data and provide the same to the microprocessor 21A for the data processing. For example, the microprocessor 21A can carry out a Fourier transformation of the CT measurement data, derive layers, layer thicknesses and layer transitions, in particular their positions in depth direction; from a depth structure r(z), extract spectrally resolved reflectivity coefficients, initialize and update depth structure model data, model spectrally resolved model reflectivity coefficients in a layer transition-specific manner based on at least one parameter, and optimize depth structure model data with reference to values determined for the parameters. In particular, the microprocessor 21A can be configured, for example, to adapt model reflectivity coefficients to layer transition-specific, spectrally resolved reflectivity coefficients by the microprocessor 21A calculating, for a parameter of a model layer transition, a value optimizing the adaptation. Furthermore, the evaluation unit 21 can comprise a controller for driving different components of the XCT measurement setup 1 in order to carry out desired actions during the CT measurement, such as the setting of parameters of the XUV radiation, an impingement position and an angle of incidence on the sample P as well as the reading out, accessing and/or the sending of measurement data sets of the spectrometer 15, and the processing of the measurement data sets and model data sets, for example, an identification of maxima in the detected spectra, etc.

For further details of an exemplary XCT measurement setup 1, reference is made to the publications mentioned above.

CT measurement methods are based on the fact that, for example, interferences are evaluated in the spectrum detected with the spectrometer 15. The interferences go back, on the one hand, to the position of the surface of the sample P with respect to the layer structures located deeper in the sample P and, on the other hand, to the positions of the layer structures with respect to one another. In particular, OCT/XCT evaluation methods have been developed that also allow an unambiguous reconstruction of the axial layer structure for complex interferences (see, for example, the publication mentioned above by S. Fuchs et al.). With so-called phase retrieval (PR) XCT algorithms, not only the axial structure of the sample but also the complete spectral phase can be derived by the reconstruction. The phase carries, inter alia, information, for example, about the absorption and dispersion of the materials in the sample as well as the incidence geometry and the refractive index of the dominant material of a layer. In order to derive the axial layer structure of the sample from the reflected spectrum, e.g., a Fourier transformation of the detected spectrum into the spatial space (here, e.g., the depth direction z) can be performed.

The complex reflectivities that are caused by layer transitions can be described in the form of spectrally resolved reflectivity coefficients (modeled reflectivity coefficients are exemplarily shown in FIG. 1 on the input and output device 21C). These layer transition-specific reflectivities lead to additional phase terms that can be derived from the CT measurement data and used according to the invention for adapting depth structure model data.

With reference to FIGS. 2 to 4, an exemplarily, in particular recursive, extension of the depth structure analysis of spectral CT measurement data obtained by means of OCT, here exemplarily XCT, is disclosed below. Thereby, depth structure model data are adapted to the CT measurement data with a spatial depth resolution that preferably goes beyond a resolution in the depth direction predetermined by the CT measurement data.

The depth structure analysis shown in FIG. 2 relates to the examination of an axially structured sample P with a layer structure comprising a plurality of layers as well as regions between the layers, which are respectively referred to herein as a layer transition. For example, a layer transition can be the transition between two layers of different materials A and B. Such a transition can be characterized, for example, by a gradient in the density distribution of the materials A and B in the region of the layer transition. The layer transition can extend, e.g., in an axial section of the sample P, which is not resolved by a usual CT measurement. Furthermore, a thin layer of a further material C (material C between two “interface layers”) can form in the region of the layer transition. Such a layer transition can be present between two layers of the same material (material sequence ACA) or of different materials (material sequence ACB). Such a thin (intermediate or also surface) layer can also extend in an axial section of the sample P, which is not resolved by a usual CT measurement.

FIG. 2 schematically shows the sample P, onto which XUV/HHG radiation 31 is irradiated at an angle and from which radiation 33 is reflected according to a spectral complex field reflectivity r(ω). For the CT measurement, the sample P has, with respect to the optical properties and the irradiated area, substantially two homogeneous thick layers S1, S2 as well as two homogeneous layer transitions positioned upstream of the respective layers S1, S2 in the depth direction z in the form of a thin surface layer s1 and a thin intermediate layer s2. If the refractive index changes in the depth direction z, e.g., at the transition between the layers S1 and s2, this leads to (partial) backscattering or to reflection of the incident radiation. In the sample P are schematically indicated by arrows reflections at the front and rear sides of the thin layers s1, s2.

In a sample described in a general manner, layer transitions in the form of thin layers sj are adjacent to thick layers Sj, which can be resolved directly in the OCT signal, so that the layer structure of the sample is formed by an alternating sequence of thin layers sⱼ with layer thicknesses dⱼ and thick layers Sⱼ with layer thicknesses Dⱼ. The layer transitions represent interfaces with a reflectivity, to which reflectivity coefficients rⱼ(ω) can be assigned. The spectral complex field reflectivity r(ω) of a sample with n layers is given by the sum of the mutually independent reflectivities rⱼ(ω) in the associated depths zⱼ and the real parts of the wave number k_{z}(ω):

$$r ( ω ) = {\sum }_{j = 1}^{n} {r}_{j} ( ω ) \cdot exp [ 2 i {k}_{z} ( ω ) {z}_{j} ]$$

As is schematically summarized in FIG. 2 on the left-hand half for the known CT analysis, the intensity reflectivity R(ω) (also referred to herein as an OCT/XCT signal) is measured in the XCT measurement 101 with the spectrometer 15. The spectral complex field reflectivity r(ω) can be derived from the intensity reflectivity R(ω), e.g., by means of the phase recovery method already mentioned (step 103). In the OCT/XCT signal, there is already included the information about the depth structure r(z) (resulting purely from a CT analysis) (as explained above, the depth structure r(z) is a distribution of the complex reflectivity of the sample (derived from the measurement) along the depth direction z), which can be calculated, e.g., by Fourier transformation of the spectral complex field reflectivity r(ω) of the sample P (step 105). Furthermore, taking into account dispersion information 41 (for example, the present knowledge of the materials of the thick layers S1, S2 of the layer structure of the sample P), the z-positions of the thick layers S1, S2 and their layer thicknesses D1, D2 can be derived (step 107). These parameters essentially represent a result 43 of known OCT evaluation methods (also referred to herein as a “pure OCT depth structure (parameter)”).

For the resolution of layer transitions in the layer structure of the sample P, generally the obtaining of layer transition-specific information, the inventors used that layer transitions, which are not resolved in the pure OCT depth structure, are characterized by parameters such as a layer transition-specific roughness, a very low thickness in the nanometer range, a layer transition-specific material composition (e.g., stoichiometric ratio of the materials AB or the formation of an oxide as material C), etc. These parameters determine the layer transition-specific reflectivities rⱼ(ω) and can be used for modeling the layer transitions.

An exemplary procedure for adapting depth structure model data M to the CT measurement data of the sample P with modeling of layer transitions is schematically shown on the right-hand half of FIG. 2.

The z-positions of the layer transitions (in FIG. 2 the thin layers s1, s2), which are arranged upstream of the respective layers S1, S2 in the depth direction z, can be derived from the depth structure r(z). For these layer transitions, layer transition-specific, spectrally resolved reflectivity coefficients r_{1_exp}(ω), r_{2_exp}(ω), r_{3_exp}(ω), etc. can furthermore be extracted from the depth structure r(z), or the spectral complex field reflectivity r(ω) (step 109). For example, a depth range Δz1, which is assigned to a thin layer s1, can be identified in the depth structure r(z). In general, the processor can detect maxima in the depth structure r(z), e.g., by sequential spatial filtering of the depth structure r(z), in other words, for example, carry out an algorithm-based determination/derivation of depth ranges Δz1, Δz2 around respectively one signal peak in the depth structure r(z) (see also FIG. 4). By a Fourier transformation of the depth structure (field reflectivity) r(z) limited to the respective layer transition sj, the processor can calculate the (layer transition-specific) spectrally resolved reflectivity coefficients r_{j_exp}(ω) assigned to the layer transition.

Based on the result 43 from the evaluation of the XCT signal and the reflectivity coefficients r_{j_exp}(ω), a depth structure model is formed (step 111). The depth structure model models the layer structure of the sample P and is provided to the processor P in the form of depth structure model data M. In the example of FIG. 2, an initialization of the depth structure model for the (thick) layers S1, S2, which are derived from the depth structure r(z), is performed based on the layer thicknesses D1, D2 and the (known) layer materials assigned to the thick layers S1, S2.

Furthermore, the depth structure model comprises model layer transitions msⱼ, which are characterized by spectrally resolved model reflectivity coefficients r_{j_mod}(ω). The model reflectivity coefficients r_{j_mod}(ω) can be modeled in a layer transition-specific manner based on at least one parameter. In FIG. 2, for this purpose, roughness parameters σ1, σ2 and thicknesses d1, d2 are indicated exemplarily as exemplary parameters for the modeling of the model layer transitions ms₁, ms₂. Examples of further parameters of model layer transitions comprise a spectral reflection and/or transmission value, a material density, a material type, a material composition, a stoichiometric ratio of a material composition, and/or a material transition gradient of a material composition. For example, for a model layer transition, a sample material (such as an oxide layer) can be derived from the measured reflectivity.

FIG. 2 further shows how, in an exemplary recursive optimization, the depth structure model data M are adapted layer by layer in the depth direction z with respect to the layer transitions s1, s2 to the CT measurement data.

If, for example, a surface layer is given on the sample P, reflectivity coefficients r1_exp(ω) can be calculated for an input-side interface of the sample P, as shown in FIG. 2. Correspondingly, a model reflectivity coefficient r_{1_mod}(ω), which corresponds to the input-side interface of the sample P, can be modeled based on the parameters layer thickness d1, material type, and roughness σ1. The model reflectivity coefficient r_{1_mod}(ω) characterizes the first modelable layer transition ms₁, in particular a modelable surface layer of the sample P. Approaches for modeling the reflectivity of a surface layer are known, see also the following explanations for modeling an intermediate layer. In a first step 121 of recursively adapting the initialized depth structure model data M to the CT measurement data, the model reflectivity coefficient r_{1_mod}(ω) is optimized by matching with the reflectivity coefficient r_{1_exp}(ω) and setting values for the parameter/parameters. FIG. 2 exemplarily shows optimized parameters σ1 and d1 as result 51 of the first step 121, which are included in the depth structure model data M (update the latter) and form the basis of the calculation of a model reflectivity coefficient r_{2_mod}(ω) with respect to the propagation of the radiation up to the model layer transition ms₂.

In a second step 123 of recursively adapting the initialized depth structure model data M to the CT measurement data, a model reflectivity coefficient r_{2_mod}(ω), which corresponds to the layer s2 of the sample P, is modeled using the parameters layer thickness d2, material type, and roughness σ2. The model reflectivity coefficient r_{2_mod}(ω) characterizes the second modelable layer transition ms₂. Approaches for modeling the reflectivity of such an intermediate layer, for example, via the reflectivities of the front side and the rear side of the intermediate layer as well as their distance are known, see, for example, “Material-specific imaging of nanolayers using extreme ultraviolet coherence tomography”, F. Wiesner et al., Optica, 8(2):230-238, 2021. Refractive indices and susceptibilities of participating materials are known, for example, from “Low-energy x-ray interaction coefficients: photoabsorption, scattering, and reflection: E=100-2000 ev z=1-94”, B. L. Henke et al., Atomic data and nuclear data tables 27(1):1-144, 1982. Further modeling way, for example, for gradual material transitions between two layers is known from “Influence of surface and interface roughness on x-ray and extreme ultraviolet reflectance: A comparative numerical study”, Y. Esashi et al., OSA Continuum, 4(5):1497-1518, 2021. Further methods for reflection simulation/modeling are known, for example, from “Surface studies of solids by total reflection of x-rays”, L. G. Parratt, Physical review, 95(2):359, 1954, or “A simulation toolkit for Id ultrafast dynamics in condensed matter”, D. Schick et al., Computer Physics Communications, 185(2):651-660, 2014.

Again, a matching of in this case the model reflectivity coefficient r_{2_mod}(ω) with the reflectivity coefficient r_{2_exp}(ω) is performed for setting values for the parameters of the second modelable layer transition ms₂. FIG. 2 exemplarily shows optimized parameters σ2 and d2 as result 53 of the second step 123. These are again included in the depth structure model data M and form, together with the optimized parameters σ1 and d1, the basis of a calculation of a model reflectivity coefficient of a subsequent layer transition (generally all subsequent layer transitions), in particular with respect to the propagation of the radiation up to the subsequent model layer transition, and on the matching thereof with a correspondingly experimentally determined reflectivity coefficient (step 125). The procedure is performed for all identified layer transitions, such that the depth structure model data M are expanded layer by layer by parameters of the model layer transitions.

In other words, before adapting a model reflectivity coefficient r_{j_mod}(ω) of a layer transition j, the model reflectivity coefficients r_{k<j_mod}(ω), which precede respectively in the depth direction z, of the preceding layer transitions k (k≤j) are adapted to the layer transition-specific, spectrally resolved reflectivity coefficients r_{k<j_exp}(ω)—stepwise in the depth direction z.

Furthermore, FIG. 2 illustrates a continuous verification (step 127) of the depth structure model data M, in particular of the modeled parameters 55 (illustrated in FIG. 2 with parameters ai and di) with the superordinate information from the measured depth structure r(z). The verification causes, for example, an adaptation of the thickness D1 in view of the modeled layer thicknesses d1. The modeled layer thicknesses d1, d2 of the layer transitions are below the resolution of the CT measurement, but can be taken into account in this manner in the reconstruction of the layer structure of the sample P.

In summary, the knowledge of the layer transition-specific, spectrally resolved reflectivity coefficients r_{i_exp}(ω), obtained from the depth structure r(z), enables a selective optimization of parameters during the modeling of layer transitions in the depth direction z.

FIG. 3 shows a schematic flow diagram for illustrating an exemplary calculation of a parameter of a model layer transition during the optimization of a spectrally resolved model reflectivity coefficient. The calculation of the parameter values takes place embedded in the sequential, recursive optimization for each layer transition, for example, in a subroutine executed with the processor.

Starting from the XCT evaluation (result 43) of the CT measurement data (including phase reconstruction), the reflectivity coefficients r_{j_exp}(ω) of the layer transitions, for example, the reflectivity coefficients of individual thin oxide layers, are known. Parameter values of a modeled layer transition j, for example, the thickness dj and the roughness σj of an individual layer (or further parameters such as a material density, etc.), are now to be calculated. The depth structure model data M are used in a modeling 61 of the reflectivity coefficients r_{j_mod}(ω). Thus, e.g., the transmission of the radiation through the sample P up to the layer transition j, i.e., “above” the layer transition, is calculated according to the already optimized parameters of the depth structure model. Analytical formulas for modeling the reflection of a thin layer with reflection coefficients at the front and rear side of the thin layer taking into consideration polarization, components of the wave vector in layers above and below, angle of incidence as well as roughness (e.g., taken into consideration by the Nevot-Croce factor), etc., are known.

The subroutine shown in FIG. 3 concludes with a comparison of the reflectivity coefficient r_{j_exp}(ω) obtained from the measurement with the modeled reflectivity coefficient r_{j_mod}(ω), wherein the modeled reflectivity coefficient r_{j_mod}(ω) is varied with the help of a parameter scan 63 of the parameters modeling the layer transition j. For the evaluation of the comparison, for example, a deviation function such as the quadratic deviation X² normalized with the measurement error α(ω) can be minimized within the scope of the parameter scan 63. Alternatively, for example, absolute or logarithmic deviations can be used.

The reflection is repeated for a plurality of combinations of the parameters dj, σj to be determined until a minimum in the deviation function is reached. For the calculation, for example, the parameter space can be discretized, such that a plurality of parameter combinations can be calculated and the minimum of the deviation function can be searched for in an automated manner. Alternatively, optimization algorithms such as Downhill-Simplex, Levenberg-Marquardt, simulated annealing and genetic algorithms can be used to determine the parameters. The subroutine of FIG. 3 outputs the parameters 65 optimized for the layer transition j.

FIG. 4 illustrates the results which were obtained with the computer-implemented method described herein for adapting depth structure model data to CT measurement data within the scope of a non-destructive characterization of a metallic layer system examined by means of XCT. The sample P schematically shown in FIG. 4 comprises an intermetallic Al2Cu substrate 71 and a solid Al coating 73. In addition to the quantification of a depth profile 75 of the sample P with nanometer resolution in the depth direction z, a reconstruction of layer thickness parameters and roughness parameters of layer transitions, in this case of a surface oxide layer 77 and an interfacial oxide layer 79, was performed using the novel model-based step-by-step optimization explained herein.

In FIG. 4, reflectivity coefficients |r_{j_exp}| measured for the two layer transitions are furthermore represented via the energy E (or ω) as a solid line 81 and the associated error intervals 83 with the help of dotted lines. In the middle energy range represented, there is a large overlap with the modeled reflectivity coefficients |r_{j_mod}| in front of dashed lines.

In FIG. 4, a table 91 is furthermore represented with the result values of the sample parameters of the layer transitions/intermediate layers (thicknesses and roughnesses in nanometers) obtained within the scope of the expanded structure analysis from the non-destructive XCT measurement. The resolution of the result values lies in the range of a few nanometers and is significantly improved compared to the resolution of the pure OCT analysis. In addition, result values of surface-sensitive measurement methods, exemplarily an electron microscopic measurement (TEM) and an atomic force microscopic measurement (AFM), are furthermore listed in the table 91 for the validation of the sample parameters obtained.

It can be seen that the non-destructive XCT measurement has resolved the layer structure (Al layer thickness D1, thickness d1 of the surface oxide layer, thickness d2 of the oxide layer at the substrate interface) as well as the composition (oxides) with essentially the same spatial resolution as electron microscopy (see TEM values). Additionally, the non-destructive XCT measurement has enabled a determination of a roughness Rq0 of the outer interface and a roughness Rq1 of the inner interface with the help of the expanded structure analysis. The values of the roughnesses are comparable to the AFM values.

It is explicitly emphasized that all features disclosed in the description and/or the claims are to be regarded as separate and independent of each other for the purpose of the original disclosure as well as for the purpose of limiting the claimed invention independent of the feature combinations in the embodiments and/or the claims. It is explicitly stated that all range specifications or specifications of groups of units disclose any possible intermediate value or subgroup of units for the purpose of the original disclosure as well as for the purpose of limiting the claimed invention, in particular also as a limit of a range specification.

### LIST OF REFERENCE NUMERALS

XCT measurement setup 1
radiation source 3
source chamber 5
beam path 7
toroidal mirror 9
sample chamber 11
aluminum and zirconium membranes 13
spectrometer 15
cylindrical mirror 17
1200-line grating 19
evaluation unit 21
microprocessor 21A
memory element 21B
input and output device 21C
XUV/HHG radiation 31
reflected radiation 33
dispersion information 41
result 43
result 51, 53,
modeled parameter 55
modeling 61
parameter scan 63
optimized parameter 65
Al2Cu substrate 71
α-Al solution 73
depth profile 75
surface oxide layer 77
interfacial oxide layer 79
solid line 81
error intervals 83
modeled reflectivity coefficient 84
table 91
XCT measurement 101
steps 103 . . .
depth structure model data M
sample P
spectral complex field reflectivity r(ω)
intensity reflectivity R(ω)
layer transition-specific, spectrally resolved reflectivity coefficients r_{1_exp}(ω), r_{2_exp}(ω),
model reflectivity coefficient r_{1_mod}(ω), r_{2_mod}(ω)
depth structure r(z)
thick layers S1, S2
layer thicknesses D1, D2
layer transitions s1, s2
surface layer s1
thin intermediate layer s2
thicknesses d1, d2
roughness parameters σ1, σ2
roughness Rq0, Rq1
deviation function such as the quadratic deviation X²
depth direction z
depth ranges Δz1, Δz2

1. Computer-implemented method for adapting depth structure model data to coherence tomography measurement data of a sample, which has a plurality of layers of layer materials in a depth direction in a layer structure, comprising the steps:
providing CT measurement data in a processor, wherein the CT measurement data comprise a depth structure unambiguously derived from a spectral complex field reflectivity, wherein the depth structure is based on a resolution in the depth direction, which is predetermined by a CT measurement on which the spectral complex field reflectivity is based;
deriving from the depth structure a plurality of layers having layer thicknesses and a plurality of layer transitions, which are arranged upstream of the respective layers in the depth direction;
extracting layer transition-specific, spectrally resolved reflectivity coefficients from the depth structure for the plurality of layer transitions;
providing depth structure model data in the processor, wherein the depth structure model data model the layer structure of the sample and are initialized for the plurality of derived layers based on
the layer thicknesses and the layer materials belonging to the layers; and
a plurality of model layer transitions, the spectrally resolved model reflectivity coefficients of which can be modeled in a layer transition-specific manner based on at least one parameter;
adapting the model reflectivity coefficients to the respective layer transition-specific, spectrally resolved reflectivity coefficients by calculating a value optimizing the adaptation in each case for the at least one parameter of one of the model layer transitions; and
adapting the depth structure model data on the basis of the value calculated for the at least one parameter.

2. The computer-implemented method according to claim 1, wherein, before adapting a model reflectivity coefficient of a layer transition, the model reflectivity coefficients preceding in each case in the depth direction are adapted to the layer transition-specific, spectrally resolved reflectivity coefficients.

3. The computer-implemented method according to claim 1,
wherein the depth structure model data are adapted layer by layer in the depth direction with respect to the layer transitions; and/or
wherein the value calculated for the at least one parameter is below the resolution in the depth direction predetermined by the CT measurement data.

4. The computer-implemented method according to claim 1, wherein, for adapting the depth structure model data to the CT measurement data, the model reflectivity coefficients are recursively optimized in a plurality of steps with respect to layer transitions located deeper in the sample,
wherein, in particular, starting from the surface of the sample, the layer structure is reconstructed and the depth structure model data are updated layer by layer by
in a first step, parameters of a surface layer such as a roughness and a thickness of the surface layer are optimized by minimizing a deviation between measured and modeled reflectivity coefficients of the surface layer by varying the parameters,
the values of the parameters of the surface layer are fixed in the depth structure model data for the further optimization;
in a second step, parameters of the second layer transition in the depth direction such as a roughness and a thickness of a buried oxide layer are optimized by minimizing a deviation function between measured and modeled reflectivity coefficients of the second layer transition by varying the parameters while the parameter of the surface layer being fixed;
the values of the parameters of the second layer transition are fixed in the depth structure model data for the further optimization;
in further steps, the parameters of the layer transition respectively closest in the depth direction are successively optimized; and
optionally, the sum of the thicknesses determined for the surface layer and the subsequent layer transitions and the thicknesses of the layers determined from the depth structure is kept constant with respect to the depth structure.

5. The computer-implemented method according to claim 1, wherein layer transition-specific, spectrally resolved reflectivity coefficients are extracted by
identifying a depth region in the depth structure assigned to a layer transition, in particular by sequential spatial filtering of the depth structure; and
calculating the layer transition-specific, spectrally resolved reflectivity coefficients by a Fourier transformation of the field reflectivity limited to the respective layer transition.

6. The computer-implemented method according to claim 1, wherein the at least one parameter of a model reflectivity coefficient of a layer transition is selected from the group of parameters comprising
a layer transition thickness;
a roughness;
a spectral reflection or transmission value;
a material density;
a material type;
a material composition;
a stoichiometric ratio of a material composition; and/or
a material transition gradient of a material composition.

7. The computer-implemented method according to claim 1, wherein the layer transition comprises
an interface or an intermediate layer delimited by two interfaces between two layers of the same material; or
an interface or an intermediate layer delimited by two interfaces between two layers of different materials.

8. The computer-implemented method according to claim 1, further comprising:
a first step of adapting the initialized depth structure model data to the coherence tomography measurement data, comprising:
optimizing a model reflectivity coefficient, which is assigned to an input-side interface of the sample and represents a first modelable layer transition, in particular a modelable surface layer, by matching with a reflectivity coefficient; and/or
an output step, in which the adapted depth structure model data and in particular the set values of the at least one parameter for the layer transitions are output by the processor on an input and output device.

9. The computer-implemented method according to claim 1, wherein the value optimizing the adaptation is calculated for a layer transition by, within the scope of a parameter scan, minimizing a deviation function, which is dependent on the at least one parameter, between layer transition-specific, spectrally resolved reflectivity coefficients assigned to the layer transition and the model reflectivity coefficient assigned to the layer, in particular iteratively.

10. Method for examining a sample, which has a plurality of layers in a layer structure in a depth direction, using optical coherence tomography, comprising the steps:
performing a CT measurement on the sample, wherein a spectral complex field reflectivity is generated as CT measurement data by
a phase-sensitive measurement and evaluation of an optical coherence signal or
a spectrally resolved measurement, performed in particular within the scope of an artifact-free coherence tomography data analysis, of an intensity reflectivity of the sample and a derivation of phase information by an iterative phase retrieval method;
deriving an unambiguous depth structure by Fourier transforming the spectral complex field reflectivity using a processor, wherein the depth structure is based on a resolution in the depth direction, which is predetermined by the CT measurement;
deriving layers and layer thicknesses of the sample taking into account a dispersion correction based on known layer materials of the sample;
generating depth structure model data using the processor, wherein the depth structure model data model the layer structure of the sample and are initialized for the plurality of derived layers based on
the layer thicknesses and the layer materials belonging to the layers; and
a plurality of model layer transitions, the spectrally resolved model reflectivity coefficients of which can be modeled in a layer transition-specific manner based on at least one parameter; and
performing the computer-implemented method of claim 1 for adapting the depth structure model data to the CT measurement data of the sample, whereby the depth structure model data are expanded in particular by parameters of model layer transitions.

11. The method according to claim 10, wherein the CT measurement on the sample is performed using XUV radiation; and
in particular, the spectral width of the XUV radiation limits a resolution of the CT measurement in the depth direction to a range of a few 10 nanometers, and the computer-implemented method identifies structures assigned to the transition layers with a resolution in the nanometer range.

12. The method according to claim 10, wherein the CT measurement on the sample is performed for a plurality of lateral regions, in particular in order to detect lateral inhomogeneities, present in a layer, of parameters of the layer structure; and
wherein, in particular, radiation is irradiated successively onto the lateral regions, such that a spectral complex field reflectivity is respectively generated for the lateral regions of the plurality of lateral regions for performing the computer-implemented method for adapting the depth structure model data to the CT measurement data of the sample.
